(19) United States
(12) Patent Application Publication
(10) Pub. No.: US 2022/0401273 A1
(43) Pub. Date: Dec. 22, 2022

(54) ABSORBENT ARTICLE WITH CHANNELED CORE

(71) Applicant: Medline Industries, LP, Northfield, IL (US)

(72) Inventors: Derrick Roemisch, Lindenhurst, IL (US); Jacob Floski, Lincolnshire, IL (US); Morgan Uridil, Cedar Rapids, IA (US); Sohyun Sara Park, Atlanta, GA (US); Kristy Matus, Grayslake, IL (US); Kushal Basnet, Bettendorf, IA (US)

(21) Appl. No.: 17/842,579

(22) Filed: Jun. 16, 2022

Related U.S. Application Data
(60) Provisional application No. 63/211,797, filed on Jun. 17, 2021.

(51) Int. Cl. A61F 13/535; A61L 15/60; A61F 13/537

(52) U.S. Cl. CPC A61F 13/535; A61L 15/60; A61F 13/53747; A61F 2013/53051

(57) ABSTRACT

An absorbent article, a portion of which is configured to absorb fluids, has a chassis with a length greater than its width. The chassis length extends in a longitudinal direction from the back to the front of a user between first and second lateral edges and the width extends in a lateral direction substantially perpendicular to the length and between first and second longitudinal edges. The absorbent article includes a topsheet, backsheet, and an absorbent core positioned between the topsheet and backsheet. The absorbent core may have an upper core and a lower core, and the upper and/or lower cores may include one or more channels formed in the core.

### RELATED APPLICATIONS

This application claims priority to U.S. Provisional Patent Application No. 63/211,797 filed on Jun. 17, 2021, which is incorporated herein by reference.

### FIELD OF THE INVENTION

The present invention relates generally to absorbent articles and, in particular, to the use an absorbent core the article where the absorbent core has channels formed in the core.

### BACKGROUND

Millions of people of all ages suffer from incontinence of the bowel or bladder. Whether an infant, adult, or elderly person, the underlying cause of incontinence varies but the method of treatment typically involves absorbent article products. Adult incontinent briefs, disposable diapers, pull-up diapers, protective underwear and underpads can alleviate some of the emotional and physical discomfort of incontinence by absorbing and containing liquid and other discharges from the human body to prevent body and clothing soiling.

Typical absorbent articles include a topsheet facing the wearer that permits fluid exudates to pass through and a backsheet that prevents the exudates from escaping from the absorbent article. An absorbent core is positioned between the topsheet and backsheet to absorb and contain the fluid exudate. Much advancement has been made in the art since the introduction of the disposable absorbent article. However, quality care for patients and other users of absorbent articles requires that the article be capable of withstanding a considerable amount of liquid and other discharge. Most articles, though adapted to retain a heavy amount of liquid and other discharges, fall short of providing adequate collection and retention in all instances.

From time-to-time the absorbent articles known in the art, like those described above, fail in their ability to retain and keep liquid and other discharge within the absorbent core. In particular, such articles often fail to adequately distribute and absorb the exudate into the absorbent material of the absorbent core. Failure in waste retention is uncomfortable and bothersome to the user, but more so, is embarrassing and can result in unwanted attention, especially for users who are insecure about having to use the article. Accordingly, there is a need to provide an absorbent article that can better absorb and retain fluids and other discharge deposited onto the article.

### BRIEF DESCRIPTION OF THE DRAWINGS

Advantages of the invention will become apparent upon reading the following detailed description and upon reference to the drawings.

FIG. 1 is a top plan view of an absorbent article in a substantially flat un-contracted position.

FIG. 2 is an exploded perspective view of the absorbent article of FIG. 1, again in a substantially flat un-contracted position.

FIG. 3 is a perspective view of an upper absorbent core of an absorbent article as depicted in FIG. 1.

FIG. 4 is a perspective view of a lower absorbent core of an absorbent article as depicted in FIG. 1.

FIG. 5 is a cross-section of an end view of a portion of an absorbent article.

FIG. 6 is a cross-section of a side view of a portion of the absorbent article of in FIG. 5.

FIG. 7 is a cross sectional view of an upper absorbent core.

FIG. 8 is a plan view of the upper absorbent core of FIG. 5

FIG. 9 is a cross sectional view of an upper absorbent core.

FIG. 10 is a plan view of the upper absorbent core of FIG. 5

FIG. 11 is a cross sectional view of an upper absorbent core.

FIG. 12 is a plan view of the upper absorbent core of FIG. 5

FIG. 13 is a cross sectional view of an upper absorbent core.

FIG. 14 is a plan view of the upper absorbent core of FIG. 5

FIG. 15 is a plan view of an absorbent structure.

FIG. 16 shows plan views of absorbent structures using various upper and lower absorbent cores.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATIVE EMBODIMENTS

Embodiments of the invention are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, forward and rearward, and the like may be used solely to distinguish one entity or action from another entity or action without necessarily requiring or implying any actual such relationship, direction or order between such entities or actions.

In particular, throughout the description, reference is made to top/upper and bottom/lower absorbent cores or layers of an absorbent core. In general, the upper core or layer refers to a core or layer closer to the user as compared to a lower core or layer, and the lower core or layer refers to the core or layer farther from the user as compared to an upper core or layer. However, unless expressly indicated, this convention is only to aid in referencing the various layers and is not intended to limit the scope of the disclosed or claimed embodiments.

Absorbent articles as described herein generally include a moisture-pervious inner layer, an absorbent layer, and a moisture-impervious outer layer. Although the remainder of the description will be specifically directed to adult incontinence articles, such as disposable diapers, it is to be understood that the embodiments may also be implemented using other absorbent articles and that the properties and uses described below apply to these other absorbent articles as well. Throughout this application, the terms absorbent article and diaper are used interchangeably. However, it should be understood that the term diaper is intended to include other absorbent articles, such as training pants, incontinence pads, etc., as would be understood by one of ordinary skill in the art. The terms user and patient are also used interchangeably to indicate a person on whom a diaper is placed.

As used herein, the following terms have the following meanings:

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Channel” refers to a void, cut-out, cavity, opening or similar feature formed in an element of an absorbent article, and in particular embodiments refers to such a feature formed in a layer or core of an absorbent core or other portion of the article intended to absorb fluid. Unless expressly indicated otherwise, channels are not limited by the manner in which they may be formed or cut into the element.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Integral” is used to refer to various portions of a single unitary element rather than separate structures bonded to or placed with or placed near one another.

These terms may be defined with additional language elsewhere in the specification.

It should be observed that the embodiments reside primarily in the combinations of assembly components and method steps for using various embodiments of the absorbent articles disclosed herein. Accordingly, the assembly components and the method steps have been represented (where appropriate) by conventional symbols in the drawings, showing only those specific details that are pertinent to understanding the embodiments of the present invention so as not to obscure the disclosure with details that will be readily apparent to those of ordinary skill in the art having the benefit of the description herein.

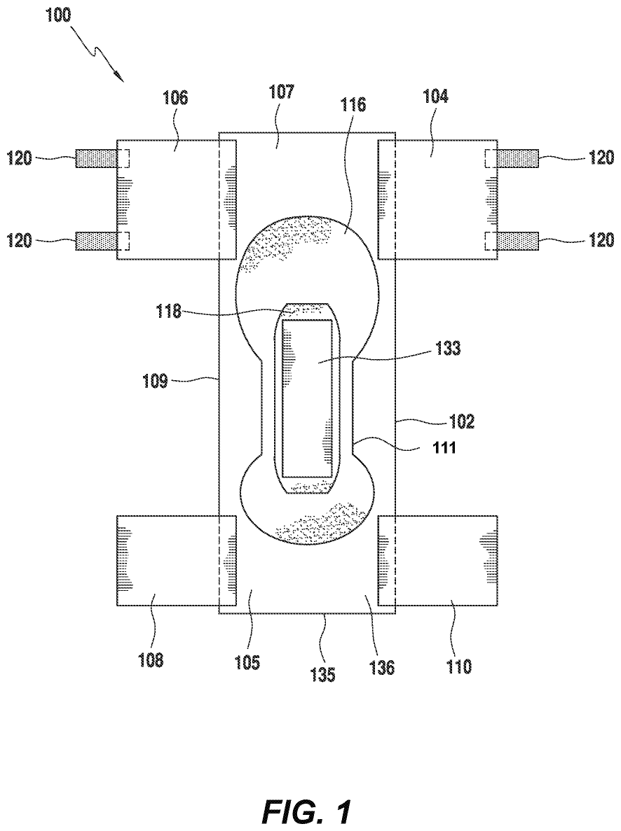

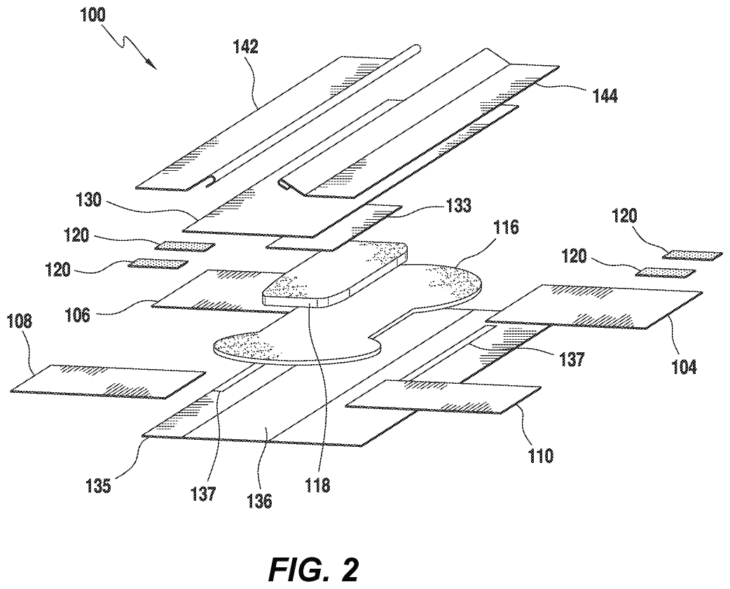

FIGS. 1 and 2 illustrate an exemplary non-limiting general embodiment of an absorbent article 100. FIG. 1 illustrates a plan view of the absorbent article 100 in a substantially flat un-contracted state. As shown in these figures, the absorbent article 100 generally consists of several layers, including an inner layer, an absorbent layer, and an outer layer. The inner layer faces a wearer and contacts the skin of the wearer when the absorbent article 100 is secured to the wearer. The inner layer may comprise a topsheet 130 that is composed of a moisture-pervious fabric suitable to allow bodily discharge to pass through the inner layer and be absorbed by the absorbent layer. Non-limiting examples of materials suitable to form the topsheet 130 include polypropylene, polyethylene, polyester, materials having hydrophobic properties, combinations thereof and/or the like. Additionally, the topsheet can be treated with a hydrophilic finish to improve pass through of liquids to diaper layers beneath the inner layer. Non-limiting examples of suitable hydrophilic finishes include stearic acid, melamine-based chemicals, fluorocarbon chemicals, and silicon-based chemicals.

The plan view of FIG. 1 is shown from the top or patient contacting side of the absorbent article. The topsheet (130) and other components have been removed for clarity. FIG. 2 is an exploded perspective view of the absorbent article 100. Again, the article 100 is shown in a substantially flat un-contracted state with certain items removed for clarity.

As shown in FIG. 1, an embodiment of the absorbent article 100 comprises a chassis 102. The chassis 102 includes a front waist region 105, a back waist region 107, and a crotch region 109 that is disposed longitudinally between the front and back waist regions 105 and 107. The front waist region 105 and the back waist region 107 generally comprise those portions of the absorbent article 100 which, when worn, encircle the waist of the wearer. The crotch region 109 is that portion of the absorbent article 100 which, when the absorbent article 100 is worn, is generally positioned between the legs of the wearer.

The chassis 102 has a shape such that its outer perimeter is rectangular or at least substantially rectangular in the illustrative embodiment of the absorbent article 100. In other embodiments, there may be portions of the chassis that are shaped and/or removed, such as in the crotch region 109, for example, resulting in a narrower crotch region portion 109 to provide a contoured fit between the legs. Still other embodiments known in the art have different shaped chassis, such as hourglass shapes, T-shapes, and the like.

Rear side panels 104, 106 are coupled to and may extend from the back waist region 107. The disposable article may further include front side panels 108, 110 that are coupled to and may extend from the front waist region 105. The back region 107 is generally positioned against the back of the user. The front waist region 105 is generally positioned against the front of the user. The rear side panels 104, 106 are configured to wrap around a wearer's waist from back to front, extending from each side of the back waist region 107. The front side panels 108, 110 are configured to wrap around a wearer's waist from front to back. In this manner, rear side panel 106 can be connected to front side panel 108 and rear side panel 104 can be connected to front side panel 110 to couple the front waist region 105 to the back waist region 107. In this embodiment there are four side panels 104, 106, 108, 110. However, other embodiments known in the art may be configured with more or fewer side panels. For instance, rear side panels 104, 106 may connect directly to an outside surface of front waist region 105 rather than to front side panels 108, 110.

The side panels may attach to the chassis 102 in a variety of manners as would be apparent to one of skill in the art. Alternatively, one or more of the side panels may be integrally formed, in whole or in part, with a backsheet 135 or topsheet 130 of the absorbent article. The backsheet 135 will have an outside surface facing away from the patient wearing the absorbent article and an inside surface 136 facing toward the patient.

The rear side panels 104, 106 may also include fasteners 120. Fasteners 120 may comprise adhesive tape, hook and loop, snaps or any other appropriate fasteners as would be understood by one of ordinary skill in the art. As shown in the illustrative embodiment, rear side panel 104, 106 includes two fasteners 120. Fasteners 120 can be configured to operatively couple rear side panels 104, 106 to a front region 105 of the diaper chassis 102. Alternative, fasteners 120 may also engage front side panels 108, 110 to attach rear side panels 104, 106, respectively. While FIG. 1 depicts rear side panels 104, 106 as including two fasteners 120, it will be understood to those skilled in the art that more or fewer fasteners may be used. While FIG. 1 depicts fasteners 120 sized and shaped a particular way, in other embodiments, fasteners 120 can be a different size and/or shape. Alternatively, the front side panels 108, 110 may include fasteners in additions to, or in place of, the fasteners 120 attached to rear side panels 104, 106.

In another embodiment, the front region 105 and/or front panels 108, 110 may include added or modified features to reinforce or increase the affinity to the fastening device. Additionally, features may be employed to allow adhesive fasteners to be attached and detached from the fastening region multiple times. Those skilled in the art will recognize that there are multiple approaches to doing so via modification of the base material as well as additions of various materials. For example, fasteners 120 may incorporate the hook component of a hook-and-loop closure and portions of the front region 105 and/or front panels 108, 110 may be comprise a corresponding loop component. The surface of front region 105 and/or front panels 108, 110 may be treated to increase or decrease the affinity for the hook components. Alternatively, separate loop component material may be adhered to the surface of the front region 105 and/or front panels 108, 110.

The absorbent article 100 may further include a set of leak guards and/or a set of leg cuffs 142, 144, both known to those of ordinary skill in the art. Additionally, the exemplary absorbent article includes an outer layer or backsheet 135 and elastic bands 137. The elastic bands 137 can be used to gather the leg of the article around the user's leg, improving the fit of the absorbent article 100 and can improve the comfort of the wearer. Elastic bands and other elastic materials may be used at other places in the absorbent article in order to improve the fit and/or fluid retention of the article.

Referring again to FIGS. 1 and 2, embodiments of the absorbent article 100 include an absorbent structure 111. The absorbent structure may comprise an acquisition and/or distribution (“A/D”) layer 133, a first or lower absorbent core 116, and a second or upper absorbent core 118.

The liquid acquisition and/or distribution layers serves to rapidly acquire and then distribute acquired liquid to an absorbent core for retention. To achieve rapid acquisition and distribution, these layers may include cellulosic fibers. These layers can include cross-linked cellulosic fibers to impart bulk and resilience to the layer, and wood pulp fibers to increase the wicking of liquid within the layer and to facilitate distribution of the liquid throughout the layer and ultimately to another layer, such as a storage layer, that is in liquid communication with the distribution layer.

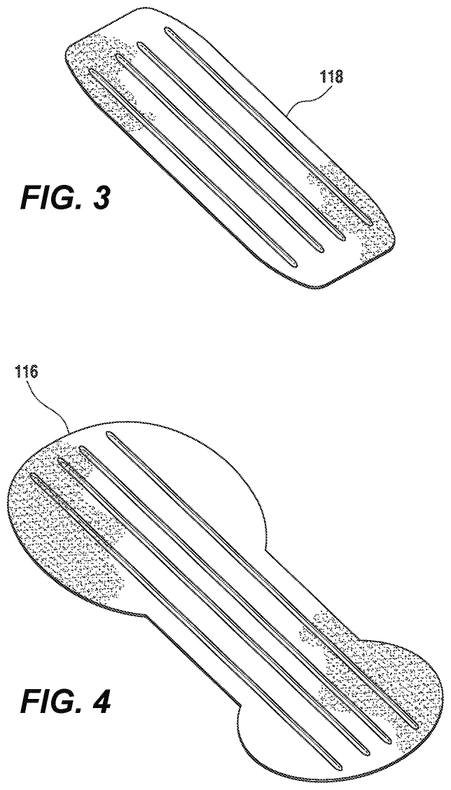

FIG. 3 is a perspective view of a top (facing towards wearer) of the upper core 118, and FIG. 4 is a perspective view of a bottom side (facing away from a wearer) of the lower core 116. Each of the lower core 116 and upper core 118 can be composed of similar material and can be shaped depending on the size of the absorbent article, and whether it is intended for use by infants, children and/or adults. By way of example, and as shown in FIGS. 3 and 4, lower core 116 can be larger and substantially hourglass shaped, whereas upper core 118 can be smaller, relative to lower core 116, and can be substantially rectangular shaped. In this manner, the absorbent article can include a large surface area of coverage provided by the lower core 116, and the increased absorbency provided by the upper core 118, without the additional bulk of an upper core having the same size as the lower core.

Lower core 116 is shown having an embossed bottom and upper core 118 is shown having an embossed top. The embossed top of upper core 118 and the embossed bottom of lower core 116 provide increased longitudinal flow while reducing lateral flow, and, in this manner, reducing leakage. Said another way, the embossed top of upper core 118 and the embossed bottom of lower core 116 allows fluid to move longitudinally towards the front and the back of a wearer, as opposed to towards the legs of a wearer.

Each of the lower core 116 and the upper core 118 may be composed of any materials suitable for absorbing the fluids and discharge including, but not limited to, a fibrous material (e.g., fluffed wood pulp), a super absorbent polymer (“SAP”), or the combination of SAP and fibrous material. The SAP can be natural or synthetic and may be biodegradable. Non-limiting examples of SAP include polymers based on acrylate(s) such as sodium acrylate, potassium acrylate, and/or an alkyl acrylate(s) (e.g., methyl acrylate, ethyl acrylate, propyl acrylate, butyl acrylate, and hexyl acrylate). The absorbency of the diaper may vary depending upon whether it is intended for use by infants, children and/or adults.

In embodiments of the invention, the lower 116 and upper 118 cores may be created with or without the use of super absorbent polymer (SAP). While some literature indicates that the presence of SAP in a diaper used for urine testing is considered benign, manufacturing diapers without SAP for the benefit of accuracy is contemplated by the present invention and may be considered beneficial.

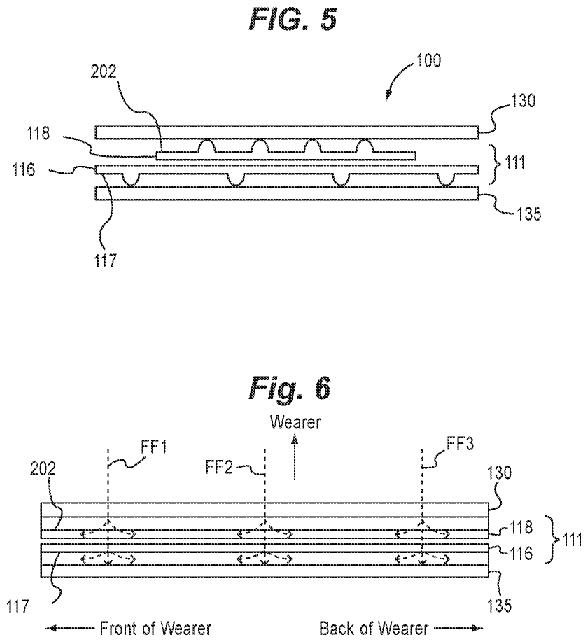

FIG. 5 is a cross-sectional end view of a portion of an absorbent article 100. The absorbent article 100 includes an inner layer 130, an absorbent structure 111, and an outer layer 135. In general, and applicable to any of the above embodiments, the absorbent structure 111 may be positioned between the inner layer 130 and the outer layer 135 of the absorbent article. The absorbent structure 111 includes a lower core 116 and an upper core 118. The lower core 116 may include an embossed bottom surface 117 and the upper core 116 may include an embossed top surface 202. In this manner, the lower core 116 and the upper core 118 provide increased longitudinal movement of fluid on both the embossed top 202 and embossed bottom 117 of the upper core 118 and the lower core 116, respectively.

FIG. 6 is a side view of absorbent article 100. As depicted in FIG. 6 fluid may flow from a wearer into contact with absorbent article 100. Fluid flow is shown in FIG. 6 as dashed paths FF1, FF2, and FF3. While fluid flow is shown as dashed paths in FIG. 6, in some embodiments, fluid may not flow along a defined path, or paths, in any pattern, and may or may not contact absorbent article at any one or multiple particular locations. The fluid can first flow into contact with, be partially absorbed by, and pass through, inner layer 130. The fluid can flow into contact with the embossed top 202 of upper core 118. A portion of the fluid can be absorbed by upper core 118 and/or move longitudinally and/or laterally on and/or within upper core 118; and a portion of the fluid can pass through upper core 118 and into contact with lower core 116. Both the greater surface area of the embossed top 202, and the space created by embossed top 202 between inner layer 130 can direct the fluid flow longitudinally towards the front and the back of the wearer, and away from edges of absorbent article 100 (e.g., reduced lateral flow. In this manner, when longitudinal flow is increased and/or when lateral flow is decreased, leakage can be reduced. A portion of the fluid can be absorbed by lower core 116 and/or move longitudinally and/or laterally on and/or within lower core 116; and a portion of the fluid can pass through lower core 116 and into contact with outer layer 135. Both the greater surface area of the embossed bottom 117, and the space created by embossed bottom 117 between outer layer 135 can direct the fluid flow longitudinally towards the front and the back of the wearer, and away from edges of absorbent article 100 (e.g., reduced lateral flow. In this manner, when longitudinal flow is increased and/or when lateral flow is decreased, leakage can be reduced.

While FIGS. 3-6 depict the lower core 116 having an embossed bottom, and the upper core 118 having an embossed top, in some embodiments, an absorbent article can have only a single core with no embossing, a single core with embossing on both, and/or other combinations of one or two cores each with embossing on one, both, or neither side. While the figures show absorbent articles include one or two cores, in some embodiments, absorbent articles can include more or fewer cores.

FIGS. 3 and 4 depict embossing as including four spaced apart embossing “lines,” in some embodiments. However, a core can include more or fewer embossing lines. In some embodiments, embossing lines can be adjacent one another, or can be a combination of adjacent and space apart embossing line. In this manner, the different combinations of embossing lines can define an embossing pattern. While FIGS. 3 and 4 depict embossing substantially along the entire width and length of each respective core, in some embodiments a core can have embossing substantially along an entire width and/or length, and/or a portion of a width and/or length.

Alternatively, in embodiments of the cores, an “accordion,” corrugated or other three-dimensional shape may be formed on faces of the cores by depositing material into a mold or onto a contoured screen using a process as shown, for example in U.S. Pat. No. 5,540,872, which is incorporated herein by reference, or as would otherwise be understood by one of ordinary skill in the art. A molded, corrugated shape may increase the flow of fluid toward the front and back of the user, as described above with regard to embossing. However, a molded shape is distinct from embossing in that it does not create lines or areas of increased density as may occur with embossing.

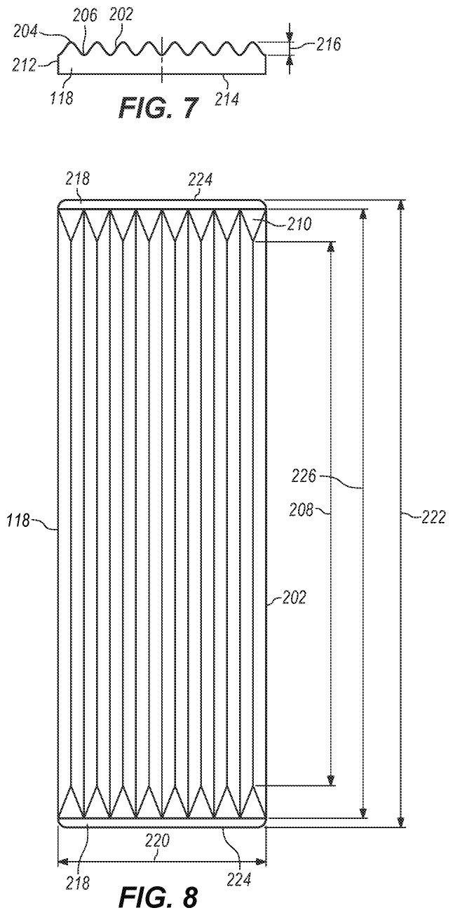

FIGS. 7 and 8 depict an embodiment of an upper core (118) of the absorbent article. The upper core may extend for a length 222 in a longitudinal direction and a width 220 in a lateral direction. A top surface 202 of the upper core may have a corrugated shape with peaks 204 and troughs 206. The corrugation peaks 204 may extend for a length 208 along a substantial portion of the upper core in the longitudinal direction and end in a tapered portion 210 where the peak tapers downwardly to the same level as the trough 206 at a greater longitudinal length 226. The upper core may have a residual thickness 212 between the trough and a bottom face 214 of the core. The residual thickness may be greater than the height 216 of the corrugations. The upper core may include a shoulder portion 218 adjacent to laterally extending edges 224. The shoulder portion may have the residual thickness.

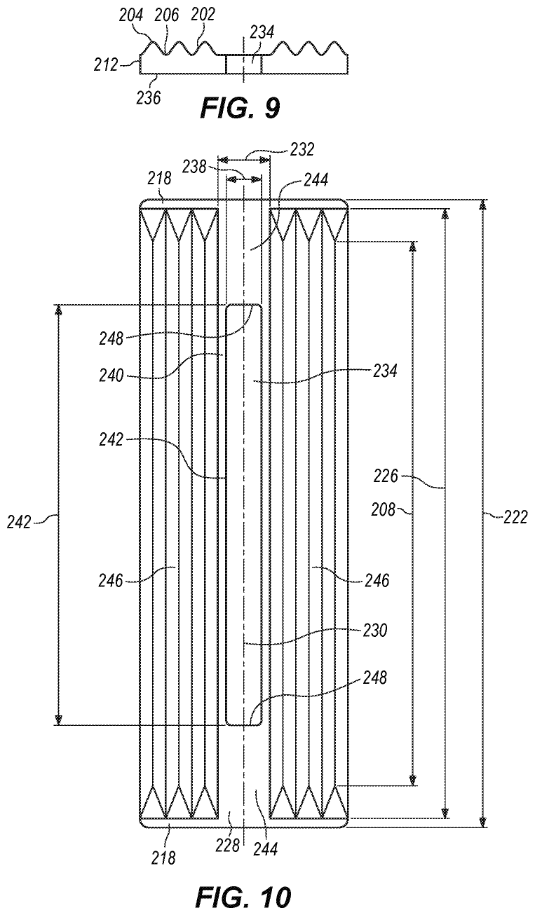

FIGS. 9 and 10 depict an embodiment of an upper core (118) with overall shape and corrugations 204, 206 similar to those described with respect to FIGS. 7 and 8. However, embodiments as illustrated in FIGS. 9 and 10 may include a valley 228 that extends from the shoulder portion 218. The valley 228 may extend the length 226 of the corrugated portion along a longitudinal axis 230 of the upper core. The valley may have a valley width 232. The upper core may further comprise a channel 234 that forms a void in the upper core.

The channel may extend through a thickness of the upper core from a bottom surface 236 to the top surface 202. The channel may have a length 242 and a width 238. The channel width 238 may be narrower than the valley width 232 such that the valley forms shoulders 240 along longitudinally extending edges 242 of the channel. The channel length 242 may be such that the valley comprises flat portions 244 adjacent to laterally extending end edges 248 of the channel and between corrugates sections 246 of the upper core. Alternatively, the channel may have the same width as the valley and/or the same length as the valley. Channels and depicted and described herein may further increase the movement of fluid within the absorbent structure and further reduce leakage and increase compacity as compared to an absorbent structure using only embossing or molded corrugations. Channels may be used in conjunction with or in place of embossing or molded corrugations.

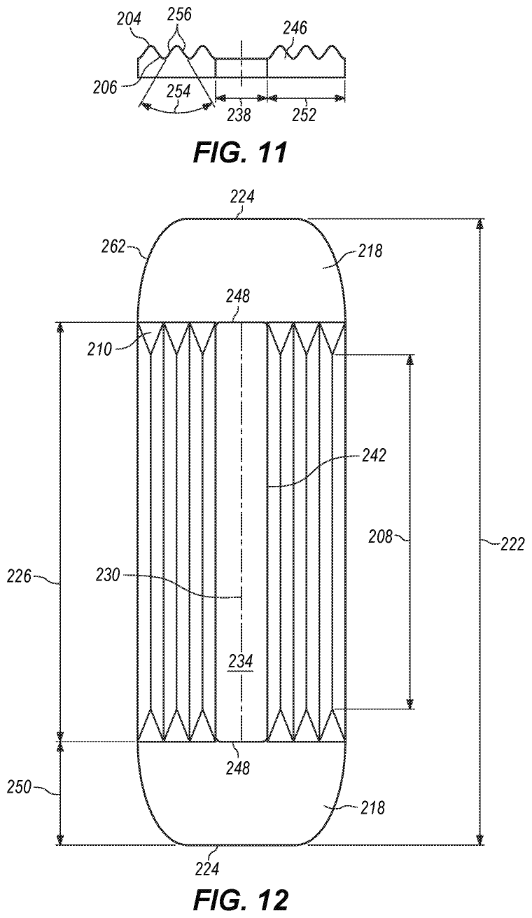

FIGS. 11 and 12 depict an embodiment of an upper core (118) with overall shape and corrugations 204, 206 with similarities to those described with respect to FIGS. 5-8. However, embodiments as illustrated in FIGS. 11 and 12 may include shoulder portions 218 that extend adjacent to laterally extending edges 224. These shoulders may extend in a longitudinal direction for a length 250 and may extend from the laterally extending end edge 224 of the core to the end edge 248 of the channel 234. In the embodiments illustrated in FIGS. 9-10, the shoulders 218 do not include any corrugations, embossing or other similar surface treatment. Additionally, in embodiments of the upper core, the channel 234 may be narrower than the valley 244 (as shown in FIG. 10) or may extend a full width between corrugated sections 246 such that there is no shoulder along longitudinally extending side edges 242 of the channel (as shown in FIG. 10). As illustrated in FIG. 12, the shoulders 218 may comprise rounded edges 262.

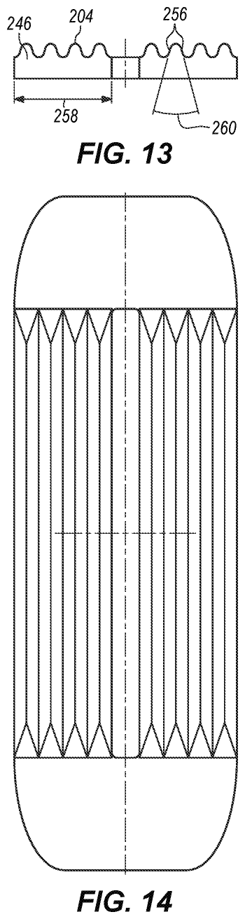

FIGS. 11 and 12 illustrate embodiments where each corrugated section 246 has a first width 252 that includes three corrugation peaks 204 in each section. Additionally, the sides 256 of each corrugation peak form an angle 254. The angle 254 may be between 45 degrees and 90 degrees and preferably may be approximately 60 degrees. Alternatively, FIGS. 13 and 14 illustrate embodiments where each corrugated section 246 has a second width 258 that includes four corrugation peaks 204 in each section. Additionally, the sides 256 of each corrugation peak form an angle 260. The angle 260 may be between 15 degrees and 45 degrees and preferably may be approximately 30 degrees.

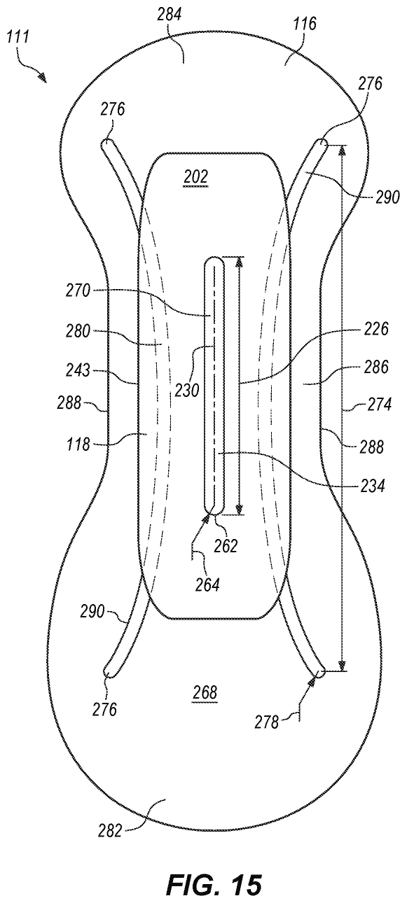

FIG. 15 illustrates an embodiment of an absorbent structure 111 with a lower core 116 and an upper core 118. The upper core may have one or more channels 234 formed in the upper core 118 and extending from a top surface 202 through the upper core 118 to a bottom surface of the upper core. The channel may have a length 226 extending generally parallel to longitudinal side edges 243 of the upper core. Ends 264 of the channel may have a radiused profile 278. The channel 234 may be positioned such that a portion 270 of the top surface 268 of the lower core 116 is accessible through the channel. The channel may allow fluid to pass through the upper core to be absorbed by the lower core.

The lower core 116 may comprise one or more channels (272) formed in the lower core 116 and extending from a top surface 268 through the core 116 to a bottom surface (117) of the lower core. The channels may extend along the lower core for a length 274. Ends 276 of the channel may have a radiused profile 278. The channels may have an arcuate or other shape such that the ends 276 of the channels are farther from a longitudinal axis 230 of the core than central portions 280 of the channels. The lower core may have an “hourglass” or “peanut” shape such that end portions 282, 284 are wider than a central portion 286 of the core. The channels may be curved such that ends 276 align with or extend farther in a lateral direction than side edges 288 of the central portion 286. The channels 272 of the lower core 116 may extend below the upper core 118 in a central portion 280 of each channel 272 and beyond the core in end portions 290 of the channel such that a bottom surface of the upper core 118 would be accessible through the channel in the central portion 280.

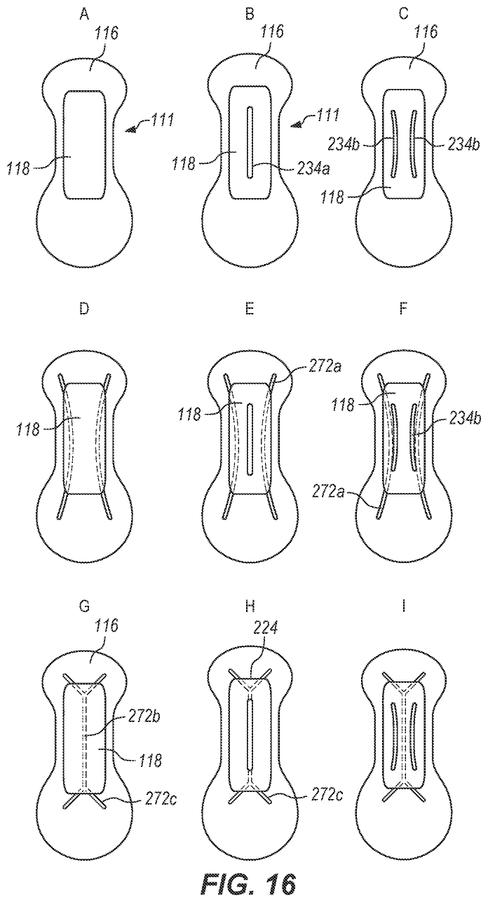

A variety of embodiments of the absorbent structure are contemplated with various combinations of channels in the upper and lower absorbent cores. Some of these configurations are illustrated in FIGS. 16A-I.

FIG. 16A illustrates embodiments of the absorbent structure 111 in which the upper core 118 does not comprise a channel, and the lower absorbent core 116 does not comprises a channel.

FIG. 16B illustrates embodiments of the absorbent structure 111 in which the lower absorbent core 116 does not comprises a channel. The upper core 118 comprises a single channel 234a. The single channel may extend linearly along a longitudinal axis of the absorbent structure. Alternatively, the single channel may extend longitudinally and/or linearly at some other position, may extend laterally, or may be angled or curved relative to the orientation of the upper core 118 and absorbent structure 111.

FIG. 16C illustrates embodiments of the absorbent structure 111 in which the lower absorbent core 116 does not comprises a channel. The upper core 118 comprises multiple channels 234b. The channels may have an arcuate shape with a center section that is closer to a longitudinal axis of the absorbent structure than are the end sections. Alternatively, the channels may extend longitudinally at some other position, may extend laterally, or may be angled or curved relative to the orientation of the upper core 118 and absorbent structure 111.

FIGS. 16D-F illustrate embodiments of the absorbent structure 111 with upper cores 118 that correspond to the upper cores of FIGS. 16A-C. FIGS. 16D-F shown a lower core 116 having at least one channel 272a. In particular, FIGS. 16D-F show a pair of arcuate channels, each with a center section that is closer to a longitudinal axis of the absorbent structure than are the end sections. Alternatively, the channels may extend longitudinally at some other position, may extend laterally, or may be angled or curved relative to the orientation of the lower core 116 and absorbent structure 111. As illustrated in FIG. 16F the channels 234b of the upper core 118 do not align with or intersect the channels 272a of the lower core 116. Alternatively, the channels may intersect at one or more points such that a portion of the channels form a through passage from a top surface of the upper core to a bottom surface of the lower core.

FIGS. 16G-I illustrate embodiments of the absorbent structure 111 with upper cores 118 that correspond to the upper cores of FIGS. 16A-C. FIGS. 16G-I shown a lower core 116 having at least one channel 272b. In particular, FIGS. 16G-I show a lower core 116 with a central channel 272b from which branch channels 272c extend at each end of the central channel. The branch channels 272 extend beyond edges 224 of the upper core 118. Alternatively, the central channel 272b may extend beyond the edges 224 of the upper core prior to branching such that branch channels 272c are completely outside the perimeter of the upper core. Alternatively, the lower core central channel 272b and branch channels 272c maybe completely covered by the upper core.

The central channel 272b may extend linearly along a longitudinal axis of the absorbent structure, and branch channels 272c extend from end points of the central channel at an angle relative to the longitudinal axis. Alternatively, the central channel may extend longitudinally and/or linearly at some other position, may extend laterally, or may be angled or curved relative to the orientation of the lower core 116 and absorbent structure 111. As illustrated in FIG. 16H, the upper core channel 234a and lower core central channel 272b may intersect or be superimposed such that a portion of the channels form a through passage from a top surface of the upper core to a bottom surface of the lower core. Alternatively, as illustrated in FIG. 161 the channels 234b of the upper core 118 do not align with or intersect the channels 272b, 272c of the lower core 116.

Corrugations or embossing of the absorbent cores, 116, 118 or their surfaces are not shown in FIG. 16. However, it should be understood that these configurations may comprise embossing, corrugations, or other surface treatments.

While the present invention has been described with reference to one or more particular embodiments, those skilled in the art will recognize that many changes may be made thereto without departing from the spirit and scope of the present invention. Furthermore, components from one embodiment can be used in other non-exclusive embodiments. By way of example, any of the absorbent articles described herein can include any of the absorbent structures described herein in relation to FIGS. 7-16, and in particular, the absorbent article 100 may include an absorbent structure having any of the various configurations shown in FIG. 16. Each of these embodiments and obvious variations thereof is contemplated as falling within the spirit and scope of the invention, which is set forth in the following alternative embodiments.

What is claimed is:

1. An absorbent structure for use in an absorbent article, the absorbent core comprising:
a lower core comprising a first lower core channel formed through the lower core extending from a lower core top surface to a lower core bottom surface; and
an upper core comprising an upper core bottom surface that is positioned adjacent to the lower core top surface, the upper core further comprising an upper core channel formed through the upper core extending from an upper core top surface to the upper core bottom surface;
wherein the upper core channel is positioned such that a portion of the lower core top surface is accessible through the upper core channel; and
wherein the first lower core channel extends beyond a periphery of the upper core.

2. The absorbent structure of claim 1, wherein the lower core further comprises a second channel formed through the lower core extending from the lower core top surface to the lower core bottom surface.

3. The absorbent structure of claim 2, wherein the second lower core channel does not intersect with the first lower core channel.

4. The absorbent structure of claim 1, wherein the upper core channel comprises a first end having a radiused profile.

5. The absorbent structure of claim 1, wherein the upper core channel extends a length that is less than a length of the upper core in a longitudinal direction.

6. The absorbent structure of claim 5, wherein the upper core channel extends in a direction parallel to a longitudinal axis of the upper core.

7. The absorbent structure of claim 1, wherein the upper core channel allows fluid to pass through the upper core to be absorbed by the lower core.

8. The absorbent structure of claim 1, wherein the first lower core channel comprises a section having an arcuate shape.

9. The absorbent structure of claim 1, wherein the first lower core channel comprises a section having an arcuate shape such that ends of the lower core channel are farther from a longitudinal axis of the lower core than is a central portion of the lower core channel.

10. The absorbent structure of claim 9, wherein the lower core further comprises a second channel formed through the lower core extending from the lower core top surface to the lower core bottom surface, and wherein the second lower core channel comprises a section having an arcuate shape.

11. The absorbent article of claim 10, wherein the second lower core channel is not parallel to the first lower core channel.

12. The absorbent article of claim 10, wherein the second lower core channel is reflectively symmetrical with the first lower core channel across a longitudinal axis of the lower core.

13. The absorbent structure of claim 1, wherein the first lower core channel and the upper core channel intersect at one or more points such that a section of the first lower core channel and a section of the upper core channel form a through passage from the upper core top surface to the lower core bottom surface.

14. The absorbent structure of claim 13, wherein the first lower core channel section is parallel the upper core channel section.

15. The absorbent structure of claim 14, wherein the first lower core channel section has a width that is the same as a width of the upper core section.

16. The absorbent structure of claim 1, wherein the lower core first channel comprises a central channel from which a first branch channel extends.

17. The absorbent structure of claim 16, wherein the branch channel extends from an end of the central channel.

18. The absorbent structure of claim 17, further comprising a second branch channel.

19. The absorbent structure of claim 18, wherein the first branch channel extends from the end of the central channel at an angle that is nonparallel to the central channel, and wherein the second branch channel extends from the end of the central channel at an angle that is nonparallel to the central channel and that is nonparallel to the first branch channel.

20. An absorbent structure for use in an absorbent article, the absorbent core comprising:
a lower core comprising a first lower core channel formed through the lower core extending from a lower core top surface to a lower core bottom surface; and
an upper core comprising an upper core bottom surface that is positioned adjacent to the lower core top surface, the upper core further comprising an upper core channel formed through the upper core extending from an upper core top surface to the upper core bottom surface;
wherein the first lower core channel and the upper core channel intersect at one or more points such that a portion of the first lower core channel and a portion of the upper core channel form a through passage from the upper core top surface to the lower core bottom surface.
